(19) United States
(12) Patent Application Publication
(10) Pub. No.: US 2022/0000573 A1
(43) Pub. Date: Jan. 6, 2022

(54) HAPTIC FEEDBACK DEVICE FOR SURGICAL INSTRUMENTS AND ROBOTIC SURGICAL SYSTEMS

(71) Applicant: Covidien LP, Mansfield, MA (US)

(72) Inventors: Osvaldo Andres Barrera, Madison, CT (US); Joe Sartor, Longmont, CO (US)

(21) Appl. No.: 17/297,136

(22) PCT Filed: Nov. 20, 2019
(86) PCT No.: PCT/US2019/062352

§ 371 Date: May 26, 2021

Related U.S. Application Data
(60) Provisional application No. 62/771,806, filed on Nov. 27, 2018.
(60) Provisional application No. 62/771,830, filed on Nov. 27, 2018.

(51) Int. Cl. A61B 34/00; A61B 34/37; A61B 90/00

(52) U.S. Cl. CPC A61B 34/76; A61B 34/37; A61B 90/06; A61B 2090/378; A61B 2090/065; A61B 2090/376; A61B 90/37

(57) ABSTRACT

A feedback patch for use with a surgical instrument or a robotic surgical system includes a substrate and a stimulator disposed on the substrate. The stimulator is configured to receive a feedback signal from a sensor of the surgical instrument or the robotic surgical system and is configured to stimulate a hand of a clinician interfacing with the surgical instrument or the robotic surgical system.

### CROSS-REFERENCE TO RELATED APPLICATIONS

The present application claims the benefit of and priority to U.S. Provisional Patent Application Ser. No. 62/771,830, filed on Nov. 27, 2018, and U.S. Provisional Patent Application Ser. No. 62/771,806, filed on Nov. 27, 2018, the entire content of each of which being incorporated herein by reference.

### BACKGROUND

Surgical instruments and robotic surgical systems have been used in surgical procedures including minimally invasive surgical procedures. During such surgical procedures, the surgical instruments or the robotic surgical system are controlled by a surgeon interfacing with a handle of the surgical instrument or a user interface of the robotic surgical system, respectively. The handle or user interface allows the surgeon to manipulate an end effector that acts on a patient. The user interface includes a handle or gimbal that is moveable by the surgeon to control a surgical robot that includes the end effector.

Surgical instruments and robotic surgical systems that lack haptic feedback may be uncomfortable and/or feel unnatural for a surgeon or cause concern for the surgeon by not allowing the surgeon to feel how the end effector is acting on tissue.

There is a need for improved feedback systems for providing haptic feedback to a surgeon interfacing with a handle of a surgical instrument or the user interface of a robotic surgical system during a surgical procedure.

### SUMMARY

In an aspect of the present disclosure, a feedback patch for use with a surgical instrument or a robotic surgical system includes a substrate and a stimulator disposed on the substrate. The stimulator is configured to receive a feedback signal from a sensor of the surgical instrument or the robotic surgical system and is configured to stimulate an arm portion of a clinician; e.g., a hand, wrist, forearm, arm, or adjacent area of a clinician; interfacing with the surgical instrument or the robotic surgical system.

In aspects, the substrate is a flexible substrate that is configured to be releasably secured to the skin of a clinician. The substrate may be configured to be secured to a handle of the surgical instrument or the robotic surgical system.

In some aspects, the stimulator is configured stimulate the arm portion of the clinician. The stimulator may mechanically vibrate and/or electrically stimulate the arm portion and/or mechanically apply pressure or shear forces on the skin of the arm portion of the clinician. The stimulator may include an array of electrodes.

In particular aspects, the stimulator is configured to vary a property of the stimulation of the arm portion in response to the received feedback signal. The property may be a frequency of the stimulation, a strength of the stimulation, an amplitude of the stimulation, and/or a pattern of the stimulation.

In another aspect of the present disclosure, a surgical system includes an end effector and a feedback device in the form of a patch or a bracelet. The end effector includes a sensor that is configured to determine forces exerted on tissue by the end effector and is configured to transmit feedback signals indicative of the forces exerted on the tissue. The feedback device includes a substrate and a stimulator that is disposed on the substrate. The stimulator is configured to receive the feedback signals from the sensor and is configured to stimulate a handle of a clinician controlling the end effector.

In aspects, the surgical system includes a hand held surgical instrument that has a handle assembly. The end effector may be operably coupled to the handle assembly of the surgical instrument. The feedback device may be secured to the handle assembly of the surgical instrument.

In some aspects, the surgical system includes a robotic surgical system that has a surgical robot. The end effector may be secured to an arm of the surgical robot. The robotic surgical system may include a user console that is configured to manipulate the surgical robot. The feedback device may be secured to an input handle of the user console.

In particular aspects, the substrate is a flexible substrate that is configured to be releasably secured to the skin of a clinician. The stimulator may be configured to mechanically vibrate and/or to electrically stimulate the arm portion of the clinician.

In another aspect of the present disclosure, a method for providing feedback with a surgical system includes transmitting a feedback signal from a sensor associated with an end effector of a surgical instrument or a robotic surgical system that is indicative of forces the end effector applies to tissue, receiving the feedback signals from the sensor at a feedback device in contact with skin of a clinician manipulating the end effector, and stimulating the skin of the clinician in response to the received feedback signals.

In aspects, stimulating the skin of the clinician includes mechanically vibrating and/or electrically stimulating the skin of the clinician. The method may include manipulating the end effector with the robotic surgical system or the handheld surgical instrument to act on tissue.

According to a further aspect of the present disclosure, a feedback device for use with a surgical instrument or a robotic surgical system is provided. The feedback device includes a body; and a first stimulator disposed within the body and configured to receive a feedback signal from a sensor of the surgical instrument or the robotic surgical system, the first stimulator configured to stimulate a portion of an arm of a clinician interfacing with the surgical instrument or the robotic surgical system.

The substrate body may be in the form of a bracelet configured to fit about the portion of the arm of the clinician.

The first stimulator may be configured to provide shear forces to the portion of the arm of the clinician, to provide shear forces longitudinally along the arm of the clinician, to provide shear forces medially about the arm of the clinician, to electrically stimulate the portion of the arm of the clinician, and/or to vary a property of the stimulation of the portion of the arm in response to the received feedback signal.

The property may be at least one of a frequency of the stimulation, a strength of the stimulation, an amplitude of the stimulation, or a pattern of the stimulation.

The feedback device may further include a second stimulator disposed within the body and configured to receive a feedback signal from a sensor of the surgical instrument or the robotic surgical system. The second stimulator may be configured to stimulate the portion of the arm of the clinician interfacing with the surgical instrument or the robotic surgical system separate and distinct from the first stimulator.

The second stimulator may be a pressure cuff. The second stimulator may be configured to pulse pressure to provide feedback to the arm portion of the clinician.

According to a further aspect of the present disclosure, a surgical system is provided and includes an end effector including a sensor configured to determine forces exerted on tissue by the end effector and configured to transmit feedback signals indicative of the forces exerted on tissue; and a feedback device including and a stimulator configured to receive the feedback signals from the sensor and to stimulate a portion of an arm of a clinician controlling the end effector.

The surgical system may further include a handheld surgical instrument having a handle assembly, wherein the end effector may be operably coupled to the handle assembly of the surgical instrument.

The feedback device may be a bracelet configured to be disposed about the portion of the arm of the clinician.

The surgical system may further include a robotic surgical system having a surgical robot, wherein the end effector may be secured to an arm of the surgical robot.

The robotic surgical system may include a user console configured to manipulate the surgical robot.

The stimulator may be configured to provide shear forces to the portion of the arm of the clinician, to provide shear forces longitudinally along the arm of the clinician, and/or to provide shear forces medially about the arm of the clinician.

The stimulator may be a pressure cuff.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a surgical system including a feedback system provided in accordance with the present disclosure;

FIG. 2 is a perspective view of the surgical system of FIG. 1 with a clinician gripping a handle assembly of the surgical system and a feedback patch of the feedback system secured to skin of the clinician;

FIG. 3 is a perspective view of another surgical system including bracelet feedback device;

FIG. 4 is an enlarged view of the bracelet feedback device of FIG. 3; and

FIG. 5 is a schematic of a robotic surgical system including a feedback system provide in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closer to the clinician and the term “distal” refers to the portion of the device or component thereof that is farther from the clinician.

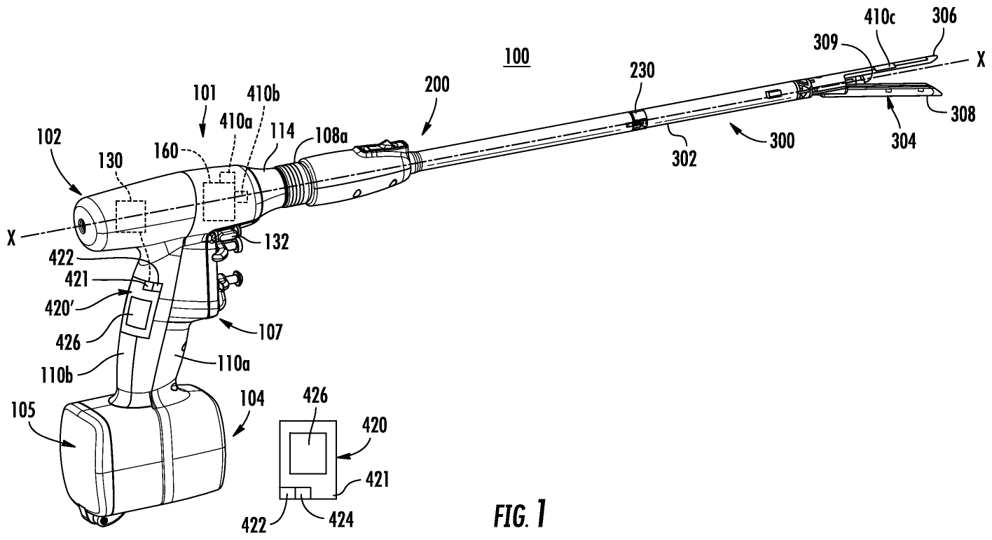

With reference to FIG. 1, a surgical system, in accordance with an embodiment of the present disclosure, is generally designated as 100, and is in the form of a powered hand held electromechanical system having a handle assembly 101 configured for selective attachment thereto of a plurality of different end effectors that are each configured for actuation and manipulation by the powered hand held electromechanical surgical system 100. The surgical system 100 includes the handle assembly 100, an adapter 200, and a loading unit 300.

The handle assembly 101 is configured for selective connection with the adapter 200, and, in turn, the adapter 200 is configured for selective connection with the end effector or loading unit 300. As detailed herein, end effector 300 is a stapling end effector; however, it is contemplated that the handle assembly 101 may be selectively connected to a plurality of end effectors that are configured to perform a variety of surgical procedures to tissue (e.g., stapling, sealing, dissecting, and sampling).

The handle assembly 101 includes a handle housing 102 having a lower housing portion 104, an intermediate housing portion 106 extending from and/or supported on lower housing portion 104, and an upper housing portion 108 extending from and/or supported on the intermediate housing portion 106. The intermediate housing portion 106 and the upper housing portion 108 are separated into a distal half-section 110a that is integrally formed with and extending from the lower portion 104, and a proximal half-section 110b connectable to the distal half-section 110a by a plurality of fasteners. When joined, the distal and proximal half-sections 110a, 110b define a handle housing 102 having a cavity therein in which a circuit board and a drive mechanism is situated.

The upper housing portion 108 of handle housing 102 provides a housing in which drive mechanism 160 is situated. The drive mechanism 160 is configured to drive shafts and/or gear components in order to perform the various operations of the surgical system 100. In particular, the drive mechanism 160 is configured to drive shafts and/or gear components in order to selectively move a tool assembly 304 of the end effector 300 relative to a proximal body portion 302 of the end effector 300, to rotate the end effector 300 about a longitudinal axis “X-X” relative to the handle housing 102, to move an anvil assembly 306 relative to a cartridge assembly 308 of the end effector 300 between open and clamped positions, or to fire a stapling and cutting cartridge within the cartridge assembly 308 of the end effector 300 to eject staples (not explicitly shown) from the cartridge assembly 308 and to advance a knife 309 through the cartridge assembly 308.

Exemplary electromechanical, hand-held, powered surgical systems and adapters are disclosed in commonly owned U.S. Pat. Nos. 8,968,276 and 9,055,943, and commonly owned U.S. Patent Publication Nos. 2015/0157321 and 2017/0296176, the entire contents of each of these disclosures are hereby incorporated by reference.

With continued reference to FIG. 1, the surgical system 100 includes a feedback system 400 provided in accordance with the present disclosure. The feedback system 400 includes a force sensor 410a-c and a feedback device or patch 420. The force sensor 410a-c is associated with the drive mechanism 160 determines the force exerted by the end effector 300 on tissue. For example, the force sensor 410a may measure a current drawn by the drive assembly 160 to determine the force applied by the end effector 300. Additionally or alternatively, the force sensor 410b may determine a torque of the drive mechanism 160. In some embodiments, the force sensor 410c is disposed in one of the jaws of the tool assembly 304 to directly measure a force applied to tissue. The force sensors 410a-c may be strain gauges, piezoelectric sensors, printed pressure-impedance sensors (flexibleforce) etc. Examples of force sensors are disclosed in U.S. patent application Ser. No. 15/887,391, filed Feb. 2, 2018, and Ser. No. 15/768,342, filed Apr. 13, 2018, and U.S. Patent Publication No. 2016/0346049, the entire contents of each of these disclosures are hereby incorporated by reference. The force sensors 410a-c may detect a pressure, a force, a bending moment, or any other force experienced by the end effector 300.

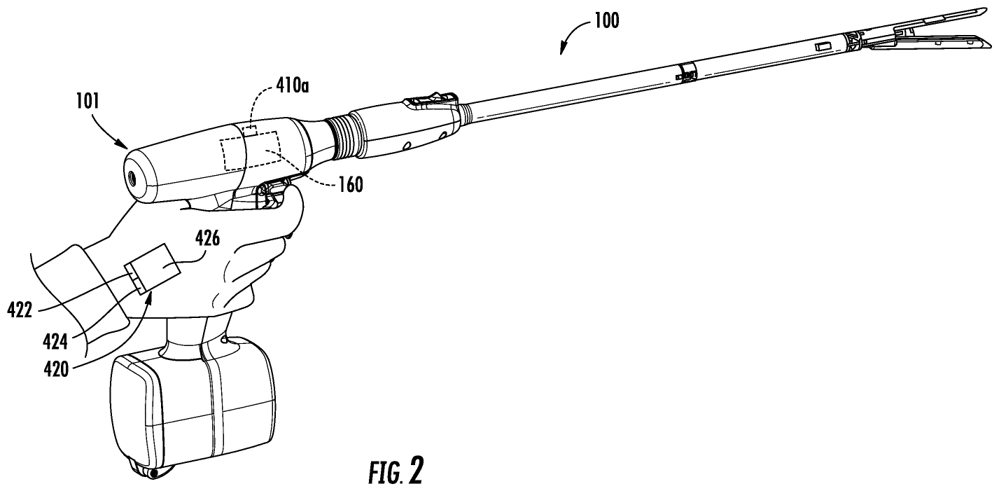

With additional reference to FIG. 2, the feedback patch 420 is secured to a portion of an arm of a clinician which may include a wrist, a forearm, an arm, or an adjacent area. As shown, the feedback patch 420 is secured to the hand of a clinician adjacent the wrist of the hand. The feedback patch 420 may be secured to a back of the hand, a palm of the hand, an arm adjacent the hand, etc. The feedback patch 420 is configured to deliver feedback signals to the clinician such that the clinician can sense forces that the end effector 300 exerts on tissue.

The feedback patch 420 includes a receiver 422, a power source 424, and a stimulator 426 secured to a flexible substrate 421. One side of the flexible substrate 421 may include an adhesive configured to releasably secure the feedback patch 420 directly to the skin of a clinician, or to a glove or garment to be worn by the clinician. Alternatively, the feedback patch 420 may be integrated into a glove or garment to be worn by the clinician. The receiver 422 is in wireless communication with one or more of the force sensors 410a-c to receive signals indicative of force delivered to tissue by the end effector 300. In some embodiments, the receiver 422 is in wired communication with the one or more force sensors 410a-c. The receiver 422 may be in direct communication with one or more of the sensors 410a-c or may be in communication with one or more of the force sensors 410a-c via a controller 130 that is disposed within the handle assembly 101. The controller 130 may be in wired or wireless communication with the force sensors 410a-c.

The power source 424 provides power to the receiver 422 and the stimulator 426. The power source 424 may be a battery, rechargeable or single use, with enough capacity to power the receiver and the stimulator 426 for one or more surgical procedures.

The stimulator 426 stimulates the clinician based on the force exerted by the end effector 300 to tissue, which is detected by one or more of the force sensors 410a-c, and which force is transmitted to the feedback patch 420. The stimulator 426 may provide vibratory stimulation or may provide electro-stimulation to the skin of the clinician. For example, the stimulator 426 may include an electrode array to provide electrical stimulation to the skin of the clinician. The stimulator is configured to vary the stimulation, e.g., vibration or electrical stimulation, by change properties of the stimulation, e.g., the frequency of stimulation, the strength or amplitude of the stimulation, the pattern of the stimulation, etc., based on the force exerted by the end effector 300 to the tissue. The properties of the stimulation may be programmable, customizable, and/or configurable based on the surgical instrument, the clinician, and/or the surgical procedure.

After a surgical procedure, the feedback patch 420 is removed from the skin of the clinician and discarded. It will be appreciated that the feedback patch 420 may be secured to a clinician underneath a sterile barrier, e.g., a sterile glove, such that the feedback patch 420 may be reused for multiple surgical procedures.

Referring back to FIG. 1, another feedback patch 420′ is provided in accordance with the present disclosure. The feedback patch 420′ is secured to the handle assembly 101 such that when a clinician grips the handle assembly 101, the feedback patch 420′ is in operable contact with the skin of the clinician such that the stimulator 426 can stimulate the clinician in response to forces exerted by the end effector 300 to tissue. The feedback patch 420′ may be in indirect contact with the skin of the clinician, through the glove worn by the clinician, such that the stimulator 426 can again stimulate the clinician in response to forces exerted by the end effector 300 to tissue. The feedback patch 420′ may include a receiver 422 and a power source 424. The stimulator 426 of the feedback patch 420 may be powered by a power source of the handle assembly 101 and/or may be in direct communication with the controller 130 such that the controller 130 controls the stimulator 426 in response to force signals received from one or more of the force sensors 410a-c.

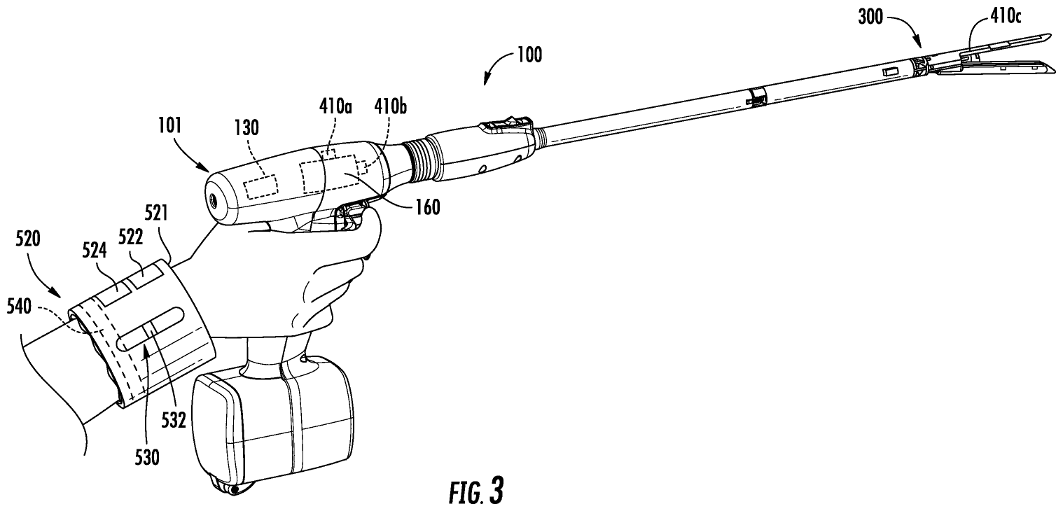

With reference to FIG. 3, another feedback device is provided in accordance with the present disclosure and is generally referred to as feedback bracelet 520. The feedback bracelet 520 is worn about a portion of an arm of a clinician. As shown, the feedback bracelet 520 includes a body 521 that forms a substantially circular arc about a portion of the arm of a clinician. The feedback bracelet 520 is worn or disposed about a forearm of the clinician; however, the feedback bracelet 520 may be worn or disposed about a wrist or another portion of the arm of the clinician. The feedback bracelet 520 is configured to deliver feedback signals to the clinician such that the clinician can sense forces that the end effector 300 exerts on tissue.

The feedback bracelet 520 includes a receiver 522, a power source 524, a first stimulator 530, and a second stimulator 540. The receiver 522 is disposed within the body 521 and is in wireless communication with one or more of the force sensors 410a-c to receive signals indicative of force delivered to tissue by the end effector 300. The receiver 522 may be in direct communication with one or more of the force sensors 410a-c or may be in communication with one or more of the force sensors 410a-c via a controller 130 that is disposed within the handle assembly 101.

The power source 524 is disposed within the body 521 and provides power to the receiver 522 and the stimulators 530, 540. The power source 524 may be a battery, rechargeable or single use, with enough capacity to power the receiver and the stimulators 530, 540 for one or more surgical procedures.

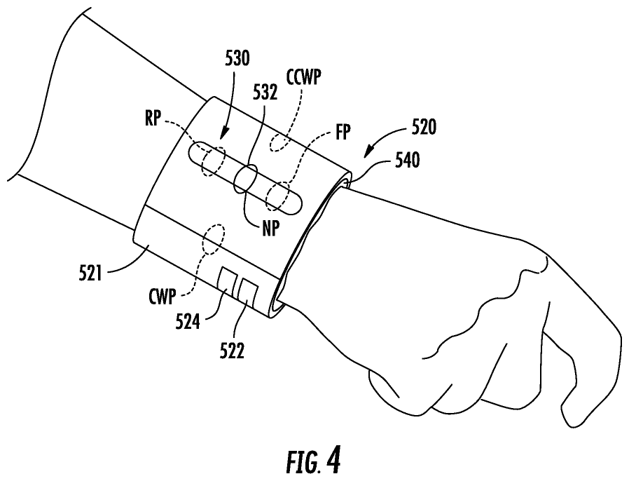

With additional reference to FIG. 4, the first stimulator 530 includes a shear actuator 532 that is disposed within the body 521 and is configured to provide shear and/or pressure forces to a portion of the arm of the clinician within the bracelet 520. The shear actuator 532 is movable “longitudinally” along a length of the arm of the clinician between a neutral position NP, a forward position FP, and a rearward position RP to provide feedback to the clinician. For example, as the end effector 300 is clamped onto tissue, the shear actuator 532 may move toward the forward position FP to indicate an increase in clamping forces within the end effector 300 and may move toward the rearward position RP to indicate a decrease in clamping forces within the end effector 300. The shear actuator 532 may also be moveable “medially” towards a counter-clockwise position CCWP and a clockwise position CWP in which the shear actuator 532 moves about an arc defined by the bracelet 520. For example, the shear actuator 532 may move toward the counter-clockwise position CCWP to indicate an increase in clamping forces within the end effector 300 and the shear actuator 532 may move towards the clockwise position CWP to indicate a decrease in clamping forces within the end effector 300. It will be appreciated that other properties and/or forces of the surgical instrument 160 and/or the end effector 300 may be indicated by movement of the shear actuator 532.

The second stimulator 540 may be a pressure cuff disposed within the body 521 and is configured to provide pressure forces to a portion of the arm of the clinician within the bracelet 520 that are separate and distinct from the feedback of the first stimulator 530. The second stimulator 540 may increase, decrease, or modulate pressure about the portion of the arm to provide feedback to the clinician. For example, the second stimulator 540 may pulse pressure as the end effector 300 is actuated or fired, e.g., staples fired from a cartridge or electrosurgical energy applied by the end effector 300. The second stimulator 540 may also be used to indicate clamping forces within the end effector 300 by increasing pressure as clamping forces within the end effector 300 increase, and decreasing pressure as clamping forces within the end effector 300 decrease.

Alternatively, the first and/or second stimulator 530, 540 may be similar to the stimulator 426 detailed above and be configured to provide vibratory stimulation or electro-stimulation to the skin of the clinician. Additionally or alternatively, the shear actuator 532 may be moved between the forward position FP and the rearward position RP to increase or reduce pressure a portion of the arm of the clinician.

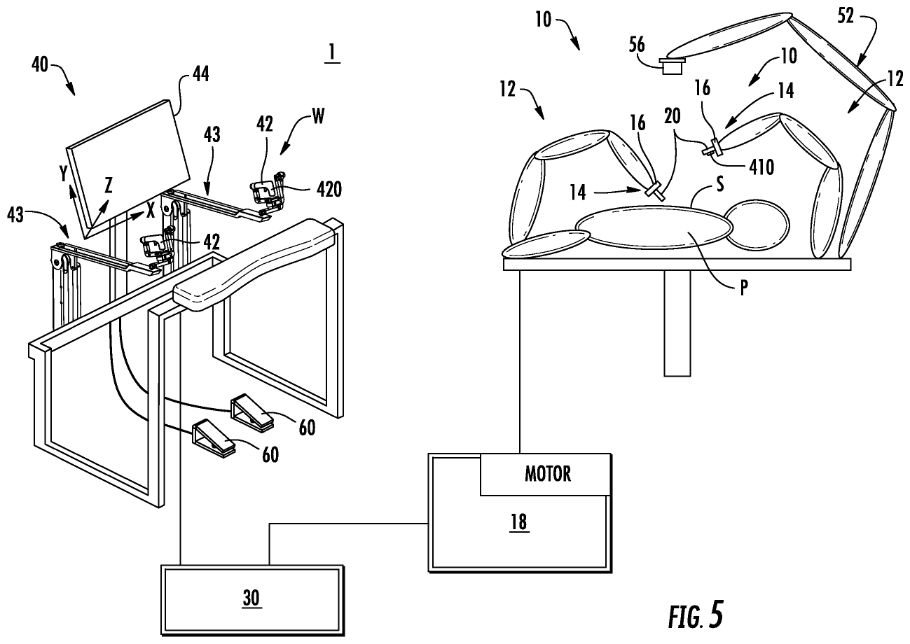

Referring now to FIG. 5, a robotic surgical system 1 in accordance with the present disclosure is shown generally as a surgical robot 10, a processing unit 30, and a user console 40. The surgical robot 10 generally includes linkages 12 and a robot base 18. The linkages 12 moveably support an end effector or tool 20 which is configured to act on tissue. The linkages 12 may be in the form of arms each having an end 14 that supports the end effector or tool 20 which is configured to act on tissue. In addition, the ends 14 of the linkages 12 may include an imaging device 16 for imaging a surgical site “S”. The user console 40 is in communication with robot base 18 through the processing unit 30.

The user console 40 includes a display device 44 which is configured to display three-dimensional images. The display device 44 displays three-dimensional images of the surgical site “S” which may include data captured by imaging devices 16 positioned on the ends 14 of the linkages 12 and/or include data captured by imaging devices that are positioned about the surgical theater (e.g., an imaging device positioned within the surgical site “S”, an imaging device positioned adjacent the patient “P”, imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S”. The imaging devices transmit captured imaging data to the processing unit 30 which creates three-dimensional images of the surgical site “S” in real-time from the imaging data and transmits the three-dimensional images to the display device 44 for display.

The user console 40 also includes input handles 42 which are supported on control arms 43 which allow a clinician to manipulate the surgical robot 10 (e.g., move the linkages 12, the ends 14 of the linkages 12, and/or the tools 20). Each of the input handles 42 is in communication with the processing unit 30 to transmit control signals thereto and to receive feedback signals therefrom. Additionally or alternatively, each of the input handles 42 may include input devices (not explicitly shown) which allow the surgeon to manipulate (e.g., clamp, grasp, fire, open, close, rotate, thrust, slice, etc.) the tools 20 supported at the ends 14 of the linkages 12.

Each of the input handles 42 is moveable through a predefined workspace to move the ends 14 of the linkages 12, e.g., tools 20, within a surgical site “S”. The three-dimensional images on the display device 44 are orientated such that the movement of the input handles 42 moves the ends 14 of the linkages 12 as viewed on the display device 44. The three-dimensional images remain stationary while movement of the input handles 42 is scaled to movement of the ends 14 of the linkages 12 within the three-dimensional images. To maintain an orientation of the three-dimensional images, kinematic mapping of the input handles 42 is based on a camera orientation relative to an orientation of the ends 14 of the linkages 12. The orientation of the three-dimensional images on the display device 44 may be mirrored or rotated relative to the view captured by the imaging devices 16, 56. In addition, the size of the three-dimensional images on the display device 44 may be scaled to be larger or smaller than the actual structures of the surgical site permitting a clinician to have a better view of structures within the surgical site “S”. As the input handles 42 are moved, the tools 20 are moved within the surgical site “S” as detailed below. Movement of the tools 20 may also include movement of the ends 14 of the linkages 12 which support the tools 20.

For a detailed discussion of the construction and operation of a robotic surgical system 1, reference may be made to U.S. Pat. No. 8,828,023, the entire contents of which are incorporated herein by reference.

As detailed above and shown in FIG. 5, the user console 40 is in operable communication with the robot system 10 to perform a surgical procedure on a patient “P”; however, it is envisioned that the user console 40 may be in operable communication with a surgical simulator (not shown) to virtually actuate a robot system and/or tool in a simulated environment. For example, the surgical robot system 1 may have a first mode where the user console 40 is coupled to actuate the robot system 10 and a second mode where the user console 40 is coupled to the surgical simulator to virtually actuate a robot system. The surgical simulator may be a standalone unit or be integrated into the processing unit 30. The surgical simulator virtually responds to a clinician interfacing with the user console 40 by providing visual, audible, force, and/or haptic feedback to clinicians through the user console 40. For example, as a clinician consoles with the input device handles 42, the surgical simulator moves representative tools that are virtually acting on tissue at a simulated surgical site. It is envisioned that the surgical simulator may allow a clinician to practice a surgical procedure before performing the surgical procedure on a patient. In addition, the surgical simulator may be used to train a clinician on a surgical procedure. Further, the surgical simulator may simulate “complications” during a proposed surgical procedure to permit a clinician to plan a surgical procedure.

With continued reference to FIG. 5, one or more of the tools 20 may include a force sensor 410 that transmits force signals indicative of the force being applied by the tool 20 to tissue. The force sensor 410 is in wired or wireless communication with the processing unit 30 to provide the force signals to the processing unit 30. The processing unit 30 transmits feedback signals to a feedback patch, e.g., feedback patch 420 (FIG. 1) that is secured to the skin of a clinician interfacing with one of the input handles 42 such that the feedback patch stimulates the skin of the clinician as detailed above or bracelet 520. Additionally or alternatively, a feedback patch 420′ may be secured to one or both of the input handles 42 to stimulate a portion of the arm of the clinician as detailed above.

By providing stimulation to the skin of a clinician, the interface with surgical instruments and/or robotic surgical systems may be more intuitive. By making a surgical instrument and/or robotic surgical system more intuitive a clinician may be more comfortable, confident, and/or efficient with the instrument or system such that a surgical procedure can be completed in less time and more efficiently, which improves the results of surgical procedures and/or reduces the costs of surgical procedures.

While a bracelet 520 is shown and described, it is further contemplated that the feedback patch may be incorporated into or onto a ring, an arm band, a head band, an ankle bracelet, or the like.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A feedback patch for use with a surgical instrument or a robotic surgical system, the feedback patch comprising:
a substrate; and
a stimulator disposed on the substrate, the stimulator configured to receive a feedback signal from a sensor of the surgical instrument or the robotic surgical system and configured to stimulate a portion of an arm of a clinician interfacing with the surgical instrument or the robotic surgical system.

2. The feedback patch according to claim 1, wherein the substrate is a flexible substrate configured to be releasably secured to the skin of a clinician.

3. The feedback patch according to claim 1, wherein the substrate is configured to be secured to a handle of the surgical instrument or the robotic surgical system.

4. The feedback patch according to claim 1, wherein the stimulator is configured to mechanically vibrate skin of the portion of the arm of the clinician.

5. The feedback patch according to claim 1, wherein the stimulator is configured to electrically stimulate the portion of the arm of the clinician.

6. The feedback patch according to claim 5, wherein the stimulator includes an array of electrodes.

7. The feedback patch according to claim 1, wherein the stimulator is configured to vary a property of the stimulation of the portion of the arm in response to the received feedback signal.

8. The feedback patch according to claim 7, wherein the property is at least one of a frequency of the stimulation, a strength of the stimulation, an amplitude of the stimulation, or a pattern of the stimulation.

9. A surgical system comprising:
an end effector including a sensor configured to determine forces exerted on tissue by the end effector and configured to transmit feedback signals indicative of the forces exerted on tissue; and
a feedback patch including a substrate and a stimulator disposed on the substrate, the stimulator configured to receive the feedback signals from the sensor and configured to stimulate a portion of an arm of a clinician controlling the end effector.

10. The surgical system according to claim 9, further comprising a handheld surgical instrument having a handle assembly, the end effector operably coupled to the handle assembly of the surgical instrument.

11. The surgical system according to claim 10, wherein the feedback patch is secured to the handle assembly of the surgical instrument.

12. The surgical system according to claim 9, further comprising a robotic surgical system including a surgical robot, the end effector secured to an arm of the surgical robot.

13. The surgical system according to claim 12, wherein the robotic surgical system includes a user console configured to manipulate the surgical robot, the feedback patch is secured to an input handle of the user console.

14. The surgical system according to claim 9, wherein the substrate is a flexible substrate configured to be releasably secured to the skin of a clinician.

15. The surgical system according to claim 9, wherein the stimulator is configured to mechanically vibrate skin of the portion of the arm of the clinician.

16. The surgical system according to claim 9, wherein the stimulator is configured to electrically stimulate the portion of the arm of the clinician.

17. A method for providing feedback with a surgical system, the method comprising:
transmitting a feedback signal from a sensor associated with an end effector of a surgical instrument or a robotic surgical system indicative of forces the end effector applies to tissue; and
receiving the feedback signals from the sensor at a feedback patch in contact with skin of a clinician manipulating the end effector; and
stimulating the skin of the clinician in response to the received feedback signals.

18. The method according to claim 17, wherein stimulating the skin of the clinician includes at least one of mechanically vibrating the skin of the clinician or electrically stimulating the skin of the clinician.

19. The method according to claim 17, further comprising manipulating the end effector to act on tissue with the robotic surgical system.

20. The method according to claim 17, further comprising manipulating the end effector to act on tissue with a handheld surgical instrument.
